(19) United States
(12) Patent Application Publication
(10) Pub. No.: US 2006/0111346 A1
(43) Pub. Date: May 25, 2006

(54) Methods of modulating high-density lipoprotein cholesterol levels and pharmaceutical formulations for the same

(75) Inventor: Jonathan M. Friedman, New York, NY (US)

Correspondence Address: PITNEY HARDIN LLP, 7 TIMES SQUARE, NEW YORK, NY, 10036-7311, US

(73) Assignee: Fazix Corporation.

(21) Appl. No.: 11/286,220

(22) Filed: Nov. 23, 2005

Related U.S. Application Data
(60) Provisional application No. 60/630,293, filed on Nov. 23, 2004.

(51) Int. Cl. A61K 31/5415

(52) U.S. Cl. 514/225.8

(57) ABSTRACT

Method of modulating High-Density Lipoprotein Cholesterol (HDL-C) levels in a mammal by administering to the mammal a therapeutically effective amount of a fluphenazine ester derivative. Pharmaceutical formulations for administration of the fluphenazine ester derivative are also disclosed.

This application claims priority under 35 U.S.C. §119(e) from Provisional Application No. 60/630,293, filed Nov. 23, 2004, which is incorporated herein by reference.

### FIELD OF THE INVENTION

The present invention relates to a method of modulating in a mammal the ratio of high-density lipoprotein cholesterol (HDL-C) to low-density lipoprotein cholesterol (LDL-C), and more particularly, to a method of increasing HDL-C levels in a mammal.

### BACKGROUND OF THE INVENTION

Cholesteryl ester transfer protein (CETP) catalyzes an essential biophysical step in the maintenance of the ratio of HDL-C to LDL-C in the blood of mammals. (Bruce C, Tall A R., Cholesteryl Ester Transfer Proteins, Reverse Cholesterol Transport, and Atherosclerosis, (1995) Curr. Opin. Lipidol. 6:306-11; LeGoff et al., Pharmacological Modulation Of Cholesteryl Ester Transfer Protein, A New Therapeutic Target In Atherogenic Dyslipidemia, (2004) Pharmacol. Ther., 101:17-38). Researchers have discovered that CETP is responsible for catalyzing the physical transfer of cholesterol esters between these two lipoprotein pools. Low values of the HDL-C to LDL-C ratio in blood have been found to correlate with increased risk of cardiovascular plaque formation and atherosclerotic disease. This increased risk is even present when the overall total cholesterol concentration is maintained at a relatively low level (e.g., in humans that do not have hypercholesterolemia). (Barter et al., Cholesteryl Ester Transfer Protein: A Novel Target For Raising HDL-C And Inhibiting Atherosclerosis, (2003) Arterioscler. Thromb. Vasc. Biol. 23:160-167). The inhibition of CETP has been postulated as a mechanism for increasing the HDL-C to LDL-C ratio thereby to reducing the risk of atherosclerotic cardiovascular plaque formation which has been associated with strokes, myocardial infarctions, and other cardiovascular or circulatory pathologies or disease conditions. (Evans et al., Medical Lipid-Regulating Therapy: Current Evidence, Ongoing Trials, Future Developments, (2004) Drugs 64:1181-1196). In fact, subsequent research has demonstrated that administration of putative CETP inhibitors has demonstrated efficacy in increasing HDL-C levels in humans. (Clark, et al., Raising High-Density Lipoproteins in Humans Through Inhibition of Cholesteryl Transfer Protein: An Initial Multidose Study of Torcetrapib, (2004) Arterioscler. Thromb. Vasc. Biol. 24:490-497).

Other CETP-targeted inhibitors have been identified (Okamoto et al., A Cholesteryl Ester Transfer Protein Inhibitor Attenuates Atherosclerosis In Rabbits, (2000) Nature 406:203-207; deGrooth et al., Efficacy And Safety Of A Novel Cholesteryl Ester Transfer Protein Inhibitor, JTT-705, In Humans, (2002) Circulation 105:2159-2165), but to date, reported measurements of inhibition of CETP activity by most of these compounds suggest only low activity in human serum. IC50 values for most of these prior compounds have all been found to be in the 1 micromolar (μM) to 10 micromolar (μM) range of concentrations. Thus, such earlier compounds reported by others exhibit expectedly limited pharmacological utility. The best known inhibitor to date exhibits an IC50 of 59 nanomolar (nM) in human serum. (Reinhard et al., Discovery Of A Simple Picomolar Inhibitor Of Cholesteryl Ester Transfer Protein, (2003) J. Med. Chem. 46:2152-2168). As a result of the low activity with candidate compounds to date, development of new inhibitors of CETP continues. (Brousseau et al., Effects Of An Inhibitor Of Cholesteryl Ester Transfer Protein On HDL Cholesterol, (2004) New Engl. J. Med. 350:1505-1515).

One of the ways in which possible candidates for CETP inhibition can be developed is through rational drug design. Rational drug design is possible through the efforts of macromolecular crystallographers who have experimentally measured the three-dimensional (3D) structures of drug target molecules or of structural homologues of such targets. One such target would be the 3D structure of bactericidal permeability-increasing protein (BPIP) (Beamer et al., Crystal Structure Of Human BPI And Two Bound Phospholipids At 2.4 Angstrom Resolution, (1997) Science 276:1861-4; Kleiger et al., The 1.7 Angstrom Crystal Structure Of BPI: A Study Of How Two Dissimilar Amino Acid Sequences Can Adopt The Same Fold, (2000) J. Mol. Biol. 299:1019-1034), a protein that bears high sequence homology and hence presumed high structural similarity to CETP. (Bruce et al., The Implications Of The Structure Of The Bactericidal Permeability-Increasing Protein On The Lipid-Transfer Function Of The Cholesteryl Ester Transfer Protein, (1998) Curr. Opin. Struct. Biol. 8:426-434; Guyard-Dangremont et al., Immunochemical Evidence That Cholesteryl Ester Transfer Protein And Bactericidal Permeability-Increasing Protein Share A Similar Tertiary Structure, (1999) Protein. Sci. 8:2392-8). From this 3D molecular structural information, workers in the field could begin to determine those molecular interactions that are the basis for enzymatic inhibitory activity. However, despite the public availability of this protein structure for several years, workers have been unable to identify compounds for which CETP inhibitory activity is acceptably high to allow general efficacious pharmacological application at such low concentrations.

In view of the above, there is a clear need in the art for compounds that exhibit modulation HDL-C to LDL-C ratios in mammals. Accordingly, an object of the present invention is to provide compounds that modulate HDL-C to LDL-C ratios upon administration to mammals.

### SUMMARY OF THE INVENTION

The present invention provides a method of modulating the level of HDL-C in a mammal. The method includes administering to the mammal a therapeutically effective amount of an ester derivative of fluphenazine having the formula (I) 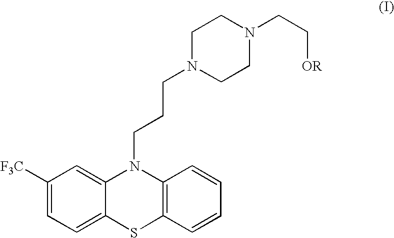
or a pharmaceutical acceptable salt thereof. In accordance with the invention, “R” is a two (2) to eighteen (18) carbon atom-containing substituent with an acyclic carbonyl-terminated linker covalently bound to the fluphenazine moiety via the carbonyl terminus. In a preferred embodiment, “R” is a substituent including five (5) to fourteen (14) carbons atoms that projects a substantially planar face. In another preferred embodiment, “R” is a substituent having a substantially planar geometry except for the acyclic carbonyl linker. Examples of substituents having a substantially planar geometry are cyclic ring structures such as a substituted or unsubstituted aromatic ring structure, a substituted or unsubstituted non-aromatic cyclic ring structure, a substituted or unsubstituted heterocyclic ring structure, or combinations thereof. Preferably, the cyclic ring structure is a monocyclic structure, a fused bicyclic structure, a fused tricyclic ring structure, or combinations thereof. The present invention also provides pharmaceutical formulations that include the above-described fluphenazine ester derivative for administration to a mammal.

Through the use of the present invention, blood serum HDL-C levels in a mammal can be dramatically increased. The modulation of HDL-C levels in mammals is particularly advantageous if the mammal is at risk of developing atherosclerotic cardiovascular plaques due to disorders such as dyslipidemia (i.e., low HDL-C levels) or hypercholesterolemia (i.e., elevated total cholesterol levels). As a result of increasing HDL-C levels in mammals at risk, the probability of developing possible life-threatening cardiovascular disease and circulatory disorders may be diminished. These and other advantages of the present invention will become more apparent from the description set forth below.

### BRIEF DESCRIPTION OF THE DRAWINGS

FIG. 1 is a timeline for experimental treatment of transgenic animals with varying doses of the fluphenazine ester derivative of the invention (FZX-CETP-001) or a placebo to assess modulation of the HDL-C to LDL-C ratio.

FIGS. 2A-2D are plot graphs depicting serum cholesterol profiles of blood sampled from Apo*E3Leiden.hCETP mice that were either a control group (-♦-) or administered with progressively increasing doses of FZX-CETP-001 via gavage (-▪-) or administered with progressively increasing doses of FZX-CETP-001 via subcutaneous injection (-▴-).

FIGS. 3A-3D are plot graphs of serum phospholipid profiles of blood sampled from Apo*E3Leiden.hCETP mice that were either a control group (-♦-) or administered with progressively increasing doses of FZX-CETP-001 via gavage (-▪-) or administered with progressively increasing doses of FZX-CETP-001 via subcutaneous injection (-▴-).

### DETAILED DESCRIPTION OF THE INVENTION

The present invention is directed to a method of modulating in a mammal the ratio of high-density-lipoprotein cholesterol (HDL-C) to low-density-lipoprotein cholesterol. In accordance with the invention, modulation of the HDL-C to LDL-C ratio is accomplished by administering to the mammal a therapeutically effective amount of an ester derivative of the neuroleptic compound, fluphenazine (also known as flufenazine). As known in the art, fluphenazine (i.e., 2-(trifluoromethyl)-10-[3-[1-(β-hydroxyethyl)-4-piperazinyl]propyl]-phenothiazine) and some of its derivatives are known for their neuroleptic properties and have been commonly administered to mammals (e.g., humans) for treatment of psychiatric disorders such as schizophrenia. It has now been discovered that certain fluphenazine esters are also effective in modulating HDL-C to LDL-C ratios in mammals.

Fluphenazine ester derivatives to be used in accordance with the invention are pharmacologically acceptable, cyclic ester derivatives represented by formula (I) as shown below: 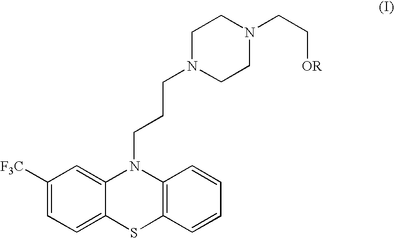
or a pharmaceutical acceptable salt thereof. In the context of the invention, “pharmacologically acceptable” means that the administered compound can be tolerated by a recipient mammal. Representative examples of pharmaceutically acceptable salts include, but are not limited to, hydrochlorides, sulfates, phosphates, ethanesulfonates, fumarates, tartrates, citrates, gluconates, and sacharinates.

In accordance with the invention, “R” is a two (2) to eighteen (18) carbon atom-containing substituent with an acyclic carbonyl-terminated linker covalently bound (i.e., bonded) to the fluphenazine moiety via the carbonyl terminus. More preferably, “R” is a substituent including five to fourteen carbons atoms that projects a substantially planar face, and more preferably has a substantially planar geometry except for the acyclic carbonyl linker.

In one preferred embodiment, “R” is a cyclic ring structure with five to fourteen carbon atoms having an acyclic carbonyl-terminated substituent (i.e., linker) bound to the fluphenazine moiety. In this embodiment, the acyclic carbonyl linker (substituted or unsubstituted) has two to four member atoms in the chain backbone. In a more preferred embodiment, the acyclic carbonyl linker has three members (including the acyl moiety) with at least one of the other two member atoms of the chain being carbon. Substituents for the acyclic carbonyl-terminated linker are hydrogen, methyl or ethyl.

The cyclic ring structures for “R” are either independently a substituted or unsubstituted aromatic ring structure, a substituted or unsubstituted non-aromatic cyclic ring structure, a substituted or unsubstituted heterocyclic ring structure, or combinations thereof. Non-aromatic cyclic ring structures include both unsaturated and alicyclic structures. Examples of ring structures for “R” include, but are not limited to, monocyclic structures (e.g., benzene, cyclohexane, piperidine, piperazine), fused bicyclic structures (e.g., naphthalene, indole, quinoline, quinoxaline, pterin) and fused tricyclic ring structures (e.g., adamantane, anthracene, indole, quinoline, quinoxaline, pterin). Substitutions on these cyclic structures (other than the carbonyl-terminated linker to the fluphenazine moiety) include, but are not limited to, C₁₋₄ alkyl groups (branched or unbranched), halogen atoms (chlorine, bromine, fluorine or iodine), nitro groups (—NO₂), C₁₋₄ alkyl ether groups (branched or unbranched) and any combination thereof. Representative examples of C₁₋₄ alkyl groups are methyl, ethyl, n-propyl, isopropyl, n-butyl, sec-butyl and tert-butyl. Representative examples of C₁₋₄ alkyl ether groups are methyl ether (i.e., methoxyl), ethyl ether, n-propyl ether, isopropyl ether, n-butyl ether, sec-butyl ether and tert-butyl ether.

Cyclic ester derivatives encompassed by formula (I) are well known in the art, such as those disclosed in British Application No.: GB 2047694, which is incorporated herein by reference. GB 2047694 discloses fluphenazine ester derivatives for neuroleptic applications encompassed by the structure of formula (I) where “R” has one of the following structures (Ha) or (IIb): 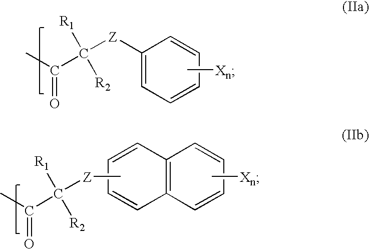
where “Z” is methylene (—CH₂—), oxygen, nitrogen or sulfur; “X” is independently a C₁₋₄ alkyl group (branched or unbranched), a halogen (chlorine, bromine, fluorine or iodine), a nitro group (—NO₂) or C₁₋₄ alkyl ether groups (branched or unbranched); and “n” is 0 to 5 for formula (IIa) and 0 to 7 for formula (IIb), with the proviso that if “n” is greater than 1, substituents “X” attached to the aromatic ring are the same or different. In addition, R₁ and R₂ are independently hydrogen, a methyl group or an ethyl group.

Representative compounds encompassed by formula (I) when “R” has the structure of formula (IIa) include, but are not limited to, fluphenazine 4-chlorophenoxyisobutyric acid ester, fluphenazine 4-iodophenoxyisobutyric acid ester, fluphenazine 4-bromophenoxyisobutyric acid ester, fluphenazine 4-fluorophenoxyisobutyric acid ester. One particularly preferred fluphenazine ester derivative is fluphenazine 4-chlorophenoxyisobutyric acid ester. However, as will be apparent to those skilled in the art, numerous other derivatives are encompassed by this structure.

Other representative compounds encompassed by formula (IIa) include, but are not limited to: fluphenazine 3,5-dimethylphenoxyisobutyric acid ester; fluphenazine phenoxyisobutyric acid ester; fluphenazine 4-methoxyphenoxyisobutyric acid ester; fluphenazine 4-dimethylaminophenoxyisobutyric acid ester; fluphenazine 2,4-dichlorophenoxyisobutyric acid ester; fluphenazine 2,6-dichlorophenoxyisobutyric acid ester; fluphenazine 3,4-dichlorophenoxyisobutyric acid ester; fluphenazine 3,5-dichlorophenoxyisobutyric acid ester; fluphenazine pentafluorophenoxyisobutyric acid ester; fluphenazine pentachlorophenoxyisobutyric acid ester; fluphenazine 3,4,5-trichlorophenoxyisobutyric acid ester; fluphenazine 4-acetylphenoxyisobutyric acid ester; fluphenazine 4-nitrophenoxyisobutyric acid ester; fluphenazine 2-nitrophenoxyisobutyric acid ester; fluphenazine 2,4-dinitrophenoxyisobutyric acid ester; fluphenazine 3,4,5-trimethoxyphenoxyisobutyric acid ester; fluphenazine 4-acetoxyphenoxyisobutyric acid ester; fluphenazine 2-acetoxyphenoxyisobutyric acid ester; fluphenazine 4-methoxyphenoxyisobutyric acid ester; fluphenazine 2-chloro-3-trifluoromethylphenoxyisobutyric acid ester; fluphenazine 2-chloro-3-trifluoromethylphenoxyisobutyric acid ester; fluphenazine 2-chloro-5-trifluoromethylphenoxyisobutyric acid ester; fluphenazine 2-fluoro-3-trifluoromethylphenoxyisobutyric acid ester; fluphenazine 2-fluoro-5-trifluoromethylphenoxyisobutyric acid ester; fluphenazine 2-phenoxypropionic acid ester; fluphenazine 2-(4-methoxyphenoxy)propionic acid ester; fluphenazine 2-(4-dimethylaminophenoxy)propionic acid ester; fluphenazine 2-(2,4-dichlorophenoxy)propionic acid ester; fluphenazine 2-(2,6-dichlorophenoxy)propionic acid ester; fluphenazine 2-(3,4-dichlorophenoxy)propionic acid ester; fluphenazine 2-(3,5-dichlorophenoxy)propionic acid ester; fluphenazine 2-(pentafluorophenoxy)propionic acid ester; fluphenazine 2-(pentachlorophenoxy)propionic acid ester; fluphenazine 2-(3,4,5-trichlorophenoxy)propionic acid ester; fluphenazine 2-(4-acetylphenoxy)propionic acid ester; fluphenazine 2-(4-nitrophenoxypropionic acid ester; fluphenazine 2-(2-nitrophenoxypropionic acid ester; fluphenazine 2-(2,4-dinitrophenoxypropionic acid ester; fluphenazine 2-(3,4,5-trimethoxyphenoxypropionic acid ester; fluphenazine 2-(4-acetoxyphenoxy)propionic acid ester; fluphenazine 2-(2-acetoxyphenoxy)propionic acid ester; fluphenazine 2-(4-methoxyphenoxy)propionic acid ester; fluphenazine 2-(4-methoxyphenoxy)propionic acid ester; fluphenazine 2-(4-methoxyphenoxy)propionic acid ester; fluphenazine 2-(2-chloro-3-trifluoromethylphenoxy)propionic acid ester; fluphenazine 2-(2-chloro-5-trifluoromethylphenoxy)propionic acid ester; fluphenazine 2-(2-fluoro-3-trifluoromethylphenoxy)propionic acid ester; and fluphenazine 2-(2-fluoro-5-trifluoromethylphenoxy)propionic acid ester.

Additional representative compounds encompassed by formula (IIa) include, but are not limited to: fluphenazine phenoxyacetic acid ester; fluphenazine 4-methoxyphenoxyacetic acid ester; fluphenazine 4-dimethylaminophenoxyacetic acid ester; fluphenazine 2,4-dichlorophenoxyacetic acid ester; fluphenazine 2,6-dichlorophenoxyacetic acid ester; fluphenazine 3,4-dichlorophenoxyacetic acid ester; fluphenazine 3,5-dichlorophenoxyacetic acid ester; fluphenazine pentafluorophenoxyacetic acid ester; fluphenazine pentachlorophenoxyacetic acid ester; fluphenazine 3,4,5-trichlorophenoxyacetic acid ester; fluphenazine 4-acetylphenoxyacetic acid ester; fluphenazine 4-nitrophenoxyacetic acid ester; fluphenazine 2-nitrophenoxyacetic acid ester; fluphenazine 2,4-dinitrophenoxyacetic acid ester; fluphenazine 3,4,5-trimethoxyphenoxyacetic acid ester; fluphenazine 4-acetoxyphenoxyacetic acid ester; fluphenazine 2-acetoxyphenoxyacetic acid ester; fluphenazine 4-methoxyphenoxyacetic acid ester; fluphenazine 4-methoxyphenoxyacetic acid ester; fluphenazine 2-chloro-3-trifluoromethylphenoxyacetic acid ester; fluphenazine 2-chloro-5-trifluoromethylphenoxyacetic acid ester; fluphenazine 2-fluoro-3-trifluoromethylphenoxyacetic acid ester; fluphenazine 2-fluoro-5-trifluoromethylphenoxyacetic acid ester; fluphenazine phenylthioisobutyric acid ester; fluphenazine 4-methoxyphenylthioisobutyric acid ester; fluphenazine 4-dimethylaminophenylthioisobutyric acid ester; fluphenazine 2,4-dichlorophenylthioisobutyric acid ester; fluphenazine 2,6-dichlorophenylthioisobutyric acid ester; fluphenazine 3,4-dichlorophenylthioisobutyric acid ester; fluphenazine 3,5-dichlorophenylthioisobutyric acid ester; fluphenazine pentafluorophenylthioisobutyric acid ester; fluphenazine pentachlorophenylthioisobutyric acid ester; fluphenazine 3,4,5-trichlorophenylthioisobutyric acid ester; fluphenazine 4-acetylphenylthioisobutyric acid ester; fluphenazine 4-nitrophenylthioisobutyric acid ester; fluphenazine 2-nitrophenylthioisobutyric acid ester; fluphenazine 2,4-dinitrophenylthioisobutyric acid ester; fluphenazine 3,4,5trimethoxyphenylthioisobutyric acid ester; fluphenazine 4-acetoxyphenylthioisobutyric acid ester; fluphenazine 2-acetoxyphenylthioisobutyric acid ester; fluphenazine 4-methoxyphenylthioisobutyric acid ester; fluphenazine 2-chloro-3-trifluoromethylphenylthioisobutyric acid ester; fluphenazine 2-chloro-3-trifluoromethylphenylthioisobutyric acid ester; fluphenazine 2-chloro-5-trifluoromethylphenylthioisobutyric acid ester; fluphenazine 2-fluoro-3-trifluoromethylphenylthioisobutyric acid ester; fluphenazine 2-fluoro-5-trifluoromethylphenylthioisobutyric acid ester; fluphenazine 2-phenylthiopropionic acid ester; fluphenazine 2-(4-methoxyphenylthio)propionic acid ester; and fluphenazine 2-(4-dimethylaminophenylthio)propionic acid ester.

Likewise, more additional representative compounds encompassed by formula (IIa) include, but are not limited to: fluphenazine 2-(2,4-dichlorophenylthio)propionic acid ester; fluphenazine 2-(2,6-dichlorophenylthio)propionic acid ester; fluphenazine 2-(3,4-dichlorophenylthio)propionic acid ester; fluphenazine 2-(3,5-dichlorophenylthio)propionic acid ester; fluphenazine 2-(pentafluorophenylthio)propionic acid ester; fluphenazine 2-(pentachlorophenylthio)propionic acid ester; fluphenazine 2-(3,4,5-trichlorophenylthio)propionic acid ester; fluphenazine 2-(4-acetylphenylthio)propionic acid ester; fluphenazine 2-(4-nitrophenylthiopropionic acid ester; fluphenazine 2-(2-nitrophenylthiopropionic acid ester; fluphenazine 2-(2,4-dinitrophenylthiopropionic acid ester; fluphenazine 2-(3,4,5-trimethoxyphenylthiopropionic acid ester; fluphenazine 2-(4-acetoxyphenylthio)propionic acid ester; fluphenazine 2-(2-acetoxyphenylthio)propionic acid ester; fluphenazine 2-(4-methoxyphenylthio)propionic acid ester; fluphenazine 2-(4-methoxyphenylthio)propionic acid ester; fluphenazine 2-(4-methoxyphenylthio)propionic acid ester; fluphenazine 2-(2-chloro-3-trifluoromethylphenylthio)propionic acid ester; fluphenazine 2-(2-chloro-5-trifluoromethylphenylthio)propionic acid ester; fluphenazine 2-(2-fluoro-3-trifluoromethylphenylthio)propionic acid ester; fluphenazine 2-(2-fluoro-5-trifluoromethylphenylthio)propionic acid ester; fluphenazine (phenylthio)acetic acid ester; fluphenazine (4-methoxyphenylthio)acetic acid ester; fluphenazine (4-dimethylaminophenylthio)acetic acid ester; fluphenazine (2,4-dichlorophenylthio)acetic acid ester; fluphenazine (2,6-dichlorophenylthio)acetic acid ester; fluphenazine (3,4-dichlorophenylthio)acetic acid ester; fluphenazine (3,5-dichlorophenylthio)acetic acid ester; fluphenazine (pentafluorophenylthio)acetic acid ester; fluphenazine (pentachlorophenylthio)acetic acid ester; fluphenazine (3,4,5-trichlorophenylthio)acetic acid ester; fluphenazine (4-acetylphenylthio)acetic acid ester; fluphenazine (4-nitrophenylthio)acetic acid ester; fluphenazine (2-nitrophenylthio)acetic acid ester; fluphenazine (2,4-dinitrophenylthio)acetic acid ester; fluphenazine (3,4,5-trimethoxyphenylthio)acetic acid ester; fluphenazine (4-acetoxyphenylthio)acetic acid ester; fluphenazine (2-acetoxyphenylthio)acetic acid ester; fluphenazine (4-methoxyphenylthio)acetic acid ester; fluphenazine (4-methoxyphenylthio)acetic acid ester; fluphenazine (2-chloro-3-trifluoromethylphenylthio)acetic acid ester; fluphenazine (2-chloro-5-trifluoromethylphenylthio)acetic acid ester; fluphenazine (2-fluoro-3-trifluoromethylphenylthio)acetic acid ester; and fluphenazine (2-fluoro-5-trifluoromethylphenylthio)acetic acid ester.

Representative compounds encompassed by formula (I) when “R” has the structure of formula (IIb) include, but are not limited to: fluphenazine 2-(6-methoxy-2-naphthyloxy)propionic acid ester, fluphenazine 2-naphthyloxyisobutyric acid ester; fluphenazine 2-heptafluoronaphthyloxyisobutyric acid ester; fluphenazine 3-methoxy-2-naphthyloxyisobutyric acid ester; fluphenazine 6-methoxy-2-naphthyloxyisobutyric acid ester; fluphenazine 7-methoxy-2-naphthyloxyisobutyric acid ester; fluphenazine 1-iodo-2-naphthyloxyisobutyric acid ester; fluphenazine 1-(dimethylaminomethyl)-2-naphthyloxyisobutyric acid ester; fluphenazine 1-(acetaminomethyl)-2-naphthyloxyisobutyric acid ester; fluphenazine 1-amino-2-naphthyloxyisobutyric acid ester; fluphenazine 1-dimethylamino-2-naphthyloxyisobutyric acid ester; fluphenazine 1-acetamino-2-naphthyloxyisobutyric acid ester; fluphenazine 3-amino-2-naphthyloxyisobutyric acid ester; fluphenazine 3-dimethylamino-2-naphthyloxyisobutyric acid ester; fluphenazine 3-acetamino-2-naphthyloxyisobutyric acid ester; fluphenazine 8-amino-2-naphthyloxyisobutyric acid ester; fluphenazine 8-dimethylamino-2-naphthyloxyisobutyric acid ester; fluphenazine 6-bromo-2-naphthyloxyisobutyric acid ester; fluphenazine 6-cyano-2-naphthyloxyisobutyric acid ester; fluphenazine 1,6-dibromo-2-naphthyloxyisobutyric acid ester; fluphenazine 1-piperidinomethyl-2-naphthyloxyisobutyric acid ester; fluphenazine 1-morpholinomethyl-2-naphthyloxyisobutyric acid ester; fluphenazine 1-naphthyloxyisobutyric acid ester; fluphenazine 1-heptafluoronaphthyloxyisobutyric acid ester; fluphenazine 4-methoxy-1-naphthyloxyisobutyric acid ester; fluphenazine 2,4-dinitro-1-naphthyloxyisobutyric acid ester; fluphenazine 2,4-dichloro-1-naphthyloxyisobutyric acid ester; fluphenazine 2-methyl-1-naphthyloxyisobutyric acid ester; fluphenazine 2-nitro-1-naphthyloxyisobutyric acid ester; fluphenazine 4-chloro-1-naphthyloxyisobutyric acid ester; fluphenazine 4-amino-1-naphthyloxyisobutyric acid ester; fluphenazine 4-dimethylamino-1-naphthyloxyisobutyric acid ester; fluphenazine 4-acetamino-1-naphthyloxyisobutyric acid ester; fluphenazine 5-amino-1-naphthyloxyisobutyric acid ester; fluphenazine 5-dimethylamino-1-naphthyloxyisobutyric acid ester; fluphenazine 5-acetamino-1-naphthyloxyisobutyric acid ester; fluphenazine 7-diethylamino-1-naphthyloxyisobutyric acid ester; and fluphenazine 7-acetamino-1-naphthyloxyisobutyric acid ester.

Additional representative compounds encompassed by “R” having the structure of formula (IIb) include, but are not limited to: fluphenazine 2-(2-naphthyloxy)propionic acid ester; fluphenazine 2-(2-heptafluoronaphthyloxy)propionic acid ester; fluphenazine 2-(3-methoxy-2-naphthyloxy)propionic acid ester; fluphenazine 2-(6-methoxy-2-naphthyloxy)propionic acid ester; fluphenazine 2-(7-methoxy-2-naphthyloxy)propionic acid ester; fluphenazine 2-(1-iodo-2-naphthyloxy)propionic acid ester; fluphenazine 2-(1-(dimethylaminomethyl)-2-naphthyloxy)propionic acid ester; fluphenazine 2-(1-(acetaminomethyl)-2-naphthyloxy)propionic acid ester; fluphenazine 2-(1-amino-2-naphthyloxy)propionic acid ester; fluphenazine 2-(1-dimethylamino-2-naphthyloxy)propionic acid ester; fluphenazine 2-(1-acetamino-2-naphthyloxy)propionic acid ester; fluphenazine 2-(3-amino-2-naphthyloxy)propionic acid ester; fluphenazine 2-(3-dimethylamino-2-naphthyloxy)propionic acid ester; fluphenazine 2-(3-acetamino-2-naphthyloxy)propionic acid ester; fluphenazine 2-(8-amino-2-naphthyloxy)propionic acid ester; fluphenazine 2-(8-dimethylamino-2-naphthyloxy)propionic acid ester; fluphenazine 2-(6-bromo-2-naphthyloxy)propionic acid ester; fluphenazine 2-(6-cyano-2-naphthyloxy)propionic acid ester; fluphenazine 2-(1,6-dibromo-2-naphthyloxy)propionic acid ester; fluphenazine 2-(1-piperidinomethyl-2-naphthyloxy)propionic acid ester; and fluphenazine 2-(1-morpholinomethyl-2-naphthyloxy)propionic acid ester;

More representative compounds encompassed by formula (IIb), include but are not limited to: fluphenazine 2-(1-naphthyloxy)propionic acid ester; fluphenazine 2-(1-heptafluoronaphthyloxy)propionic acid ester; fluphenazine 2-(4-methoxy-1-naphthyloxy)propionic acid ester; fluphenazine 2-(2,4-dinitro-1-naphthyloxy)propionic acid ester; fluphenazine 2-(2,4-dichloro-1-naphthyloxy)propionic acid ester; fluphenazine 2-(2 methyl-1-naphthyloxy)propionic acid ester; fluphenazine 2-(2-nitro-1-naphthyloxy)propionic acid ester; fluphenazine 2-(4-chloro-1-naphthyloxy)propionic acid ester; fluphenazine 2-(4-amino-1-naphthyloxy)propionic acid ester; fluphenazine 2-(4-dimethylamino-1-naphthyloxy)propionic acid ester; fluphenazine 2-(4-acetamino-1-naphthyloxy)propionic acid ester; fluphenazine 2-(5-amino-1-naphthyloxy)propionic acid ester; fluphenazine 2-(5-dimethylamino-1-naphthyloxy)propionic acid ester; fluphenazine 2-(5-acetamino-1-naphthyloxy)propionic acid ester; fluphenazine 2-(7-diethylamino-1-naphthyloxy)propionic acid ester; fluphenazine 2-(7-acetamino-1-naphthyloxy)propionic acid ester; fluphenazine 2-naphthyloxyacetic acid ester; fluphenazine 2-heptafluoronaphthyloxyacetic acid ester; fluphenazine 3-methoxy-2-naphthyloxyacetic acid ester; fluphenazine 6-methoxy-2-naphthyloxyacetic acid ester; fluphenazine 7-methoxy-2-naphthyloxyacetic acid ester; fluphenazine 1-iodo-2-naphthyloxyacetic acid ester; fluphenazine 1-(dimethylaminomethyl)-2-naphthyloxyacetic acid ester; fluphenazine 1-(acetaminomethyl)-2-naphthyloxyacetic acid ester; fluphenazine 1-amino-2-naphthyloxyacetic acid ester; fluphenazine 1-dimethylamino-2-naphthyloxyacetic acid ester; fluphenazine 1-acetamino-2-naphthyloxyacetic acid ester; fluphenazine 3-amino-2-naphthyloxyacetic acid ester; fluphenazine 3-dimethylamino-2-naphthyloxyacetic acid ester; fluphenazine 3-acetamino-2-naphthyloxyacetic acid ester; fluphenazine 8-amino-2-naphthyloxyacetic acid ester; fluphenazine 8-dimethylamino-2-naphthyloxyacetic acid ester; fluphenazine 6-bromo-2-naphthyloxyacetic acid ester; fluphenazine 6-cyano-2-naphthyloxyacetic acid ester; fluphenazine 1,6-dibromo-2-naphthyloxyacetic acid ester; fluphenazine 1-piperidinomethyl-2-naphthyloxyacetic acid ester; fluphenazine 1-morpholinomethyl-2-naphthyloxyacetic acid ester; fluphenazine 1-naphthyloxyacetic acid ester; fluphenazine 1-heptafluoronaphthyloxyacetic acid ester; fluphenazine 4-methoxy-1-naphthyloxyacetic acid ester; fluphenazine 2,4-dinitro-1-naphthyloxyacetic acid ester; fluphenazine 2,4-dichloro-1-naphthyloxyacetic acid ester; fluphenazine 2-methyl-1-naphthyloxyacetic acid ester; fluphenazine 2-nitro-1-naphthyloxyacetic acid ester; fluphenazine 4-chloro-1-naphthyloxyacetic acid ester; fluphenazine 4-amino-1-naphthyloxyacetic acid ester; fluphenazine 4-dimethylamino-1-naphthyloxyacetic acid ester; fluphenazine 4-acetamino-1-naphthyloxyacetic acid ester; fluphenazine 5-amino-1-naphthyloxyacetic acid ester; fluphenazine 5-dimethylamino-1-naphthyloxyacetic acid ester; fluphenazine 5-acetamino-1-naphthyloxyacetic acid ester; fluphenazine 7-diethylamino-1 naphthyloxyacetic acid ester; fluphenazine 7-acetamino-1-naphthyloxyacetic acid ester; fluphenazine 2-naphthylthioisobutyric acid ester; fluphenazine 2-heptafluoronaphthylthioisobutyric acid ester; fluphenazine 3-methoxy-2-naphthylthioisobutyric acid ester; fluphenazine 6-methoxy-2-naphthylthioisobutyric acid ester; fluphenazine 7-methoxy-2-naphthylthioisobutyric acid ester; fluphenazine 1-iodo-2-naphthylthioisobutyric acid ester; fluphenazine 1-(dimethylaminomethyl)-2-naphthylthioisobutyric acid ester; fluphenazine 1-(acetaminomethyl)-2-naphthylthioisobutyric acid ester; fluphenazine 1-amino-2-naphthylthioisobutyric acid ester; fluphenazine 1-dimethylamino-2-naphthylthioisobutyric acid ester; fluphenazine 1-acetamino-2-naphthylthioisobutyric acid ester; fluphenazine 3-amino-2-naphthylthioisobutyric acid ester; fluphenazine 3-dimethylamino-2-naphthylthioisobutyric acid ester; fluphenazine 3-acetamino-2-naphthylthioisobutyric acid ester; fluphenazine 8-amino-2-naphthylthioisobutyric acid ester; fluphenazine 8-dimethylamino-2-naphthylthioisobutyric acid ester; fluphenazine 6-bromo-2-naphthylthioisobutyric acid ester; fluphenazine 6-cyano-2-naphthylthioisobutyric acid ester; fluphenazine 1,6-dibromo-2-naphthylthioisobutyric acid ester; fluphenazine 1-piperidinomethyl-2-naphthylthioisobutyric acid ester; and fluphenazine 1-morpholinomethyl-2-naphthylthioisobutyric acid ester;

Likewise, other additional representative compounds encompassed by formula (IIb) include, but are not limited to: fluphenazine 1-naphthylthioisobutyric acid ester; fluphenazine 1-heptafluoronaphthylthioisobutyric acid ester; fluphenazine 4-methoxy-1-naphthylthioisobutyric acid ester; fluphenazine 2,4-dinitro-1-naphthylthioisobutyric acid ester; fluphenazine 2,4-dichloro-1-naphthylthioisobutyric acid ester; fluphenazine 2-methyl-1-naphthylthioisobutyric acid ester; fluphenazine 2-nitro-1-naphthylthioisobutyric acid ester; fluphenazine 4-chloro-1-naphthylthioisobutyric acid ester; fluphenazine 4-amino-1-naphthylthioisobutyric acid ester; fluphenazine 4-dimethylamino-1-naphthylthioisobutyric acid ester; fluphenazine 4-acetamino-1-naphthylthioisobutyric acid ester; fluphenazine 5-amino-1-naphthylthioisobutyric acid ester; fluphenazine 5-dimethylamino-1-naphthylthioisobutyric acid ester; fluphenazine 5-acetamino-1-naphthylthioisobutyric acid ester; fluphenazine 7-diethylamino-1-naphthylthioisobutyric acid ester; fluphenazine 7-acetamino-1-naphthylthioisobutyric acid ester; fluphenazine 2-(2-naphthylthio)propionic acid ester; fluphenazine 2-(2-heptafluoronaphthylthio)propionic acid ester; fluphenazine 2-(3-methoxy-2-naphthylthio)propionic acid ester; fluphenazine 2-(6-methoxy-2-naphthylthio)propionic acid ester; fluphenazine 2-(7-methoxy-2 naphthylthio)propionic acid ester; fluphenazine 2-(1-iodo-2-naphthylthio)propionic acid ester; fluphenazine 2-(1-(dimethylaminomethyl)-2-naphthylthio)propionic acid ester; fluphenazine 2-(1-(acetaminomethyl)-2-naphthylthio)propionic acid ester; fluphenazine 2-(1-amino-2-naphthylthio)propionic acid ester; fluphenazine 2-(1-dimethylamino-2-naphthylthio)propionic acid ester; fluphenazine 2-(1-acetamino-2-naphthylthio)propionic acid ester; fluphenazine 2-(3-amino-2-naphthylthio)propionic acid ester; fluphenazine 2-(3-dimethylamino-2-naphthylthio)propionic acid ester; fluphenazine 2-(3-acetamino-2-naphthylthio)propionic acid ester; fluphenazine 2-(8-amino-2-naphthylthio)propionic acid ester; fluphenazine 2-(8-dimethylamino-2-naphthylthio)propionic acid ester; fluphenazine 2-(6-bromo-2-naphthylthio)propionic acid ester; fluphenazine 2-(6-cyano-2-naphthylthio)propionic acid ester; fluphenazine 2-(1,6-dibromo-2-naphthylthio)propionic acid ester; fluphenazine 2-(1-piperidinomethyl-2-naphthylthio)propionic acid ester; fluphenazine 2-(1-morpholinomethyl-2-naphthylthio)propionic acid ester; fluphenazine 2-(1-naphthylthio)propionic acid ester; fluphenazine 2-(1-heptafluoronaphthylthio)propionic acid ester; fluphenazine 2-(4-methoxy-1-naphthylthio)propionic acid ester; fluphenazine 2-(2,4-dinitro-1-naphthylthio)propionic acid ester; fluphenazine 2-(2,4-dichloro-1-naphthylthio)propionic acid ester; fluphenazine 2-(2-methyl-1-naphthylthio)propionic acid ester; fluphenazine 2-(2-nitro-1-naphthylthio)propionic acid ester; fluphenazine 2-(4-chloro-1-naphthylthio)propionic acid ester; fluphenazine 2-(4-amino-1-naphthylthio)propionic acid ester; fluphenazine 2-(4-dimethylamino-1-naphthylthio)propionic acid ester; fluphenazine 2-(4-acetamino-1-naphthylthio)propionic acid ester; fluphenazine 2-(5-amino-1-naphthylthio)propionic acid ester; fluphenazine 2-(5-dimethylamino-1-naphthylthio)propionic acid ester; fluphenazine 2-(5-acetamino-1-naphthylthio)propionic acid ester; fluphenazine 2-(7-diethylamino-1-naphthylthio)propionic acid ester; fluphenazine 2-(7-acetamino-1-naphthylthio)propionic acid ester;

Other additional representative compounds encompassed by formula (IIb) include, but are not limited to: fluphenazine (2-naphthylthio)acetic acid ester; fluphenazine (2-heptafluoronaphthylthio)acetic acid ester; fluphenazine (3-methoxy-2-naphthylthio)acetic acid ester; fluphenazine (6-methoxy-2-naphthylthio)acetic acid ester; fluphenazine (7-methoxy-2-naphthylthio)acetic acid ester; fluphenazine (1-iodo-2-naphthylthio)acetic acid ester; fluphenazine (1-(dimethylaminomethyl)-2-naphthylthio)acetic acid ester; fluphenazine (1-(acetaminomethyl)-2-naphthylthio)acetic acid ester; fluphenazine (1-amino-2-naphthylthio)acetic acid ester; fluphenazine (1-dimethylamino-2-naphthylthio)acetic acid ester; fluphenazine (1-acetamino-2-naphthylthio)acetic acid ester; fluphenazine (3-amino-2-naphthylthio)acetic acid ester; fluphenazine (3-dimethylamino-2-naphthylthio)acetic acid ester; fluphenazine (3-acetamino-2-naphthylthio)acetic acid ester; fluphenazine (8-amino-2-naphthylthio)acetic acid ester; fluphenazine (8-dimethylamino-2-naphthylthio)acetic acid ester; fluphenazine (6-bromo-2-naphthylthio)acetic acid ester; fluphenazine (6-cyano-2-naphthylthio)acetic acid ester; fluphenazine (1,6-dibromo-2-naphthylthio)acetic acid ester; fluphenazine (1-piperidinomethyl-2-naphthylthio)acetic acid ester; fluphenazine (1-morpholinomethyl-2-naphthylthio)acetic acid ester; fluphenazine (1-naphthylthio)acetic acid ester; fluphenazine (1-heptafluoronaphthylthio)acetic acid ester; fluphenazine (4-methoxy-1-naphthylthio)acetic acid ester; fluphenazine (2,4-dinitro-1-naphthylthio)acetic acid ester; fluphenazine (2,4-dichloro-1-naphthylthio)acetic acid ester; fluphenazine (2-methyl-1-naphthylthio)acetic acid ester; fluphenazine (2-nitro-1-naphthylthio)acetic acid ester; fluphenazine (4-chloro-1-naphthylthio)acetic acid ester; fluphenazine (4-amino-1-naphthylthio)acetic acid ester; fluphenazine (4-dimethylamino-1-naphthylthio)acetic acid ester; fluphenazine (4-acetamino-1-naphthylthio)acetic acid ester; fluphenazine (5-amino-1-naphthylthio)acetic acid ester; fluphenazine (5-dimethylamino-1-naphthylthio)acetic acid ester; fluphenazine (5-acetamino-1-naphthylthio)acetic acid ester; fluphenazine (7-diethylamino-1-naphthylthio)acetic acid ester; and fluphenazine (7-acetamino-1-naphthylthio)acetic acid ester.

Other representative structures for “R” include, but are not limited to, the following structures set forth below, (IIIa), (IIIb) and (IIIc): 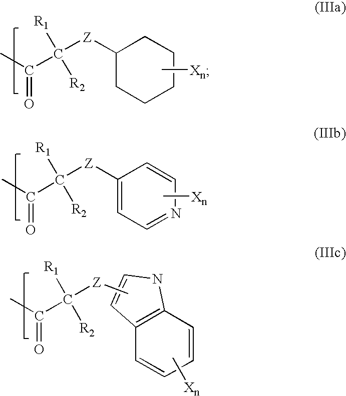
where “Z” is methylene (—CH₂—), oxygen, nitrogen or sulfur; “X” is independently a C₁₋₄ alkyl group (branched or unbranched), a halogen (chlorine, bromine, fluorine or iodine), a nitro group (—NO₂) or C₁₋₄ alkyl ether groups (branched or unbranched); and “n” is 0 to 5 for formula (IIIa), 0 to 4 for formula (IIIb), and 0 to 7 for formula (IIIc), with the proviso that if “n” is greater than 1 substituents “X” attached to the ring are the same or different. In addition, R₁ and R₂ are independently hydrogen, a methyl group or an ethyl group.

Representative compounds encompassed by formula (I) when “R” has the structure of formula (IIIa) include, but are not limited to: fluphenazine cyclohexyloxyisobutyric acid ester; fluphenazine 2-(cyclohexyloxy)propionic acid ester; and fluphenazine cyclohexyloxyacetic acid ester.

Representative compounds encompassed by formula (I) when “R” has the structure of formula (IIIb) include, but are not limited to: fluphenazine 2-(4-pyridoxy)propionic acid ester; fluphenazine 4-pyridoxyisobutyric acid ester; and fluphenazine 4-pyridoxyacetic acid ester.

Representative compounds encompassed by formula (I) when “R” has the structure of formula (IIIc) include, but are not limited to: fluphenazine 2-(indole-4-oxy)propionic acid ester; fluphenazine 2-(indole-5-oxy)propionic acid ester; fluphenazine 2-[3-(2-aminoethyl)indole-5-oxy]propionic acid ester; fluphenazine 2-[3-(2-dimethylaminoethyl)indole-5-oxy]propionic acid ester; fluphenazine indole-4-oxyisobutyric acid ester; fluphenazine indole-5-oxyisobutyric acid ester; fluphenazine 3-(2-aminoethyl)indole-5-oxyisobutyric acid ester; fluphenazine 3-(2-dimethylaminoethyl)indole-5-oxyisobutyric acid ester; fluphenazine indole-4-oxyacetic acid ester; fluphenazine indole-5-oxyacetic acid ester; fluphenazine 3-(2-aminoethyl)indole-5-oxyacetic acid ester; and fluphenazine 3-(2-dimethylaminoethyl)indole-5-oxyacetic acid ester.

In another embodiment of the invention, the fluphenazine ester derivatives to be utilized are acyclic (i.e., non-cyclic) ester derivatives of fluphenazine. Representative examples of acyclic ester derivatives of fluphenazine are also well known in the art. In a preferred embodiment, the ester moiety of the acyclic derivative is an unbranched, linear chain with possibly one or more carbons replaced with a heteroatom. While branched derivatives can also be utilized, chains with quaternary or tertiary bonded carbons with substituents having greater than two members (e.g., a propyl group) should be avoided. While branched acyclic derivatives can also be utilized, with a branched acyclic derivative having a quaternary carbon (or hetero-) atom, one should avoid hydrocarbon substituents (saturated or unsaturated) having greater than two carbon atoms (e.g., a propyl group) for at least one of the branching chains extending from this quaternary atom away from the fluphenazine moiety. This restriction is to maintain substantial planarity for this portion of the molecule. The other two substituents extending from the quaternary atom away from the fluphenazine moiety can include longer hydrocarbon substituents among others. Two well known acyclic esters are the linear esters, fluphenazine n-decanoate, and fluphenazine n-ethanoate. Other linear ester derivatives include, but are not limited to, fluphenazine n-butyroate, fluphenazine n-pentanoate, fluphenazine n-hexanoate, fluphenazine n-heptanoate, fluphenazine n-octanoate, fluphenazine n-nonoate, fluphenazine n-undecanoate, fluphenazine n-dodecanoate, fluphenazine n-tridecanoate, and fluphenazine n-tetradecanoate.

The above-described fluphenazine ester derivatives were identified as inhibitors of CETP activity (i.e., the transfer of cholesterol esters between serum HDL-C and LDL-C by CETP) based on computer modeling of the crystal structures of the catalytic domains of bactericidal permeability-increasing protein (BPIP). Based of X-ray diffraction patterns (10,11), BPIP was determined to have a highly conserved amino acid sequence domain and therefore implied structural homology to CETP. With SHOSITES analyses (Friedman, J. M., Fourier-Filtered van der Waals Surface: Accurate Ligand Shapes From Protein Structures, (1997) Protein Eng. 10:851-863), the fluphenazine esters derivatives were determined to have similar characteristic structural features that allow for binding with the active domain of CETP.

Synthesis of fluphenazine (i.e., phenothiazine) ester derivatives as set forth above can be easily synthesized by those skilled in the art following the teachings of GB 2047694. As set forth in GB 2047694, the fluphenazine esters derivatives can be synthesized by reacting fluphenazine salts or active esters with carboxylic acids of the general formula of “R” such as carboxylic acids having the formulas (IIa) and (IIb). Representative examples of carboxylic acids to be used include, but are not limited, phenoxyisobutyric acid, mono- and dimethylphenoxyisobutyric acids (such as 2,3-, 2,4-, 2,5-, 2,6- and 3,5-dimethylphenoxyisobutyric acid), mono- and di-tert.-butyl-phenoxyisobutyric acids (such as 4-tert.-butylphenoxyisobutyric acid), methyl-tert.-butyl-phenoxyisobutyric acids, mono- and dichlorophenoxyisobutyric acids (such as 4-chlorophenoxyisobutyric acid, 2,6-dichlorophenoxyisobutyric acid and 2,3-dichlorophenoxyisobuyric acid) 4-nitrophenoxyisobutyric acid, 1- and 2-naphthoxyisobutyric acids, mono- and dimethylnaphthoxyisobutyric acids, mono- and di-tert.-butyl-napthoxyisobutyric acids, methyl-tert.-butylnaphhoxyisobutyric acids and related carboxylic acids, furthermore 2-(6-methoxy-2-naphthyl)-propionic acid.

Likewise, the above-described fluphenazine ester derivatives can be synthesized by reacting fluphenazine salts or active esters with acyl halides of the general formula of “R” such as acyl halides having the formulas (IIa) and (IIb). One particularly preferred acyl halide is acyl chloride. In addition, other acyl compounds may be used to synthesize the above-described fluphenazine ester derivatives: alkali metal salts (e.g. sodium or potassium salts); acid anhydrides; acid azides; and so on. Esterification can be performed in an inert solvent, such as benzene, toluene, chloroform, dichloroethane, etc.

Other fluphenazine ester derivatives encompassed by formula (I) are also well known in the art. For example, esters of flufenazine, such as esters formed with various fatty acids are known (Yale, H. L., Sowinski, F., (1960) J. Am. Chem. Soc. 82:2039; Yale et al., (1963) J. Med. Chem. 6:347). Likewise, another known ester is an ester formed with 3,4,5-trimethoxybenzoic acid (Toldy et al., (1965) Acta Chim. Acad. Sci. Hung. 43:253). Another known fluphenazine ester derivative is an ester with a tricyclic fused ring formed with 1-adamantanecarboxylic acid (Yale, H. L., (1977) J. Med. Chem. 20:302).

As described above, the fluphenazine ester derivative is administered to a mammal in a therapeutically effective amount. A “therapeutically effective amount” is any amount that modulates a mammal's HDL-C to LDL-C ratio by providing a measurable increase in HDL-C blood plasma levels as compared to HDL-C levels prior to administration of the fluphenazine ester derivative. Although not required by the invention, a measurable decrease of LDL-C levels can occur with a concomitant increase in HDL-C blood plasma levels. While not wishing to be limited by theory, it is believed that the increase of HDL-C blood plasma levels is due to the inhibition of CETP by the above-described fluphenazine ester derivatives. In a preferred embodiment, the amount administered is an amount that increases the HDL-C blood plasma level of the mammal by at least 10 percent, with at least 20 percent being more preferred. Although from the examples set forth below, increases significantly greater than 100 percent are achievable through present invention. Moreover, in view that many fluphenazine ester derivatives are neuroleptics, it is preferred that the amount administered to the mammal is a non-neuroleptic amount (i.e., an amount that does not provide a neuroleptic effect to the recipient mammal).

The actual amount of fluphenazine ester derivative administered to a mammal will of course vary from mammal to mammal due to independent factors such as weight, age, sex, pre-existing conditions such as dyslipidemia or hypercholesterolemia, dosage formulation and dosing regimen. Preferably, the fluphenazine ester derivative is administered at least about 0.1 milligrams per kilogram of body weight (mg/kg) per day, with at least 0.3 mg/kg per day being more preferred, and with at least 1 mg/kg per day being even more preferred. In another preferred embodiment, to avoid potential neuroleptic effects, the fluphenazine ester derivative is administered at no more than 25 mg/kg per day, with less than about 20 mg/kg per day being more preferred, and with less than about 15 mg/kg per day being even more preferred.

Mammals to be administered fluphenazine ester derivatives are mammals that could benefit from a modulation of HDL-C to LDL-C blood plasma ratios. Representative mammals to be administered the fluphenazine ester derivatives are humans, simians, porcines as well as any other mammal known to express CETP. In a preferred embodiment, the mammal is a mammal in need of treatment with fluphenazine ester derivatives in accordance with the invention (i.e., the mammal requires an increase of HDL-C blood plasma levels). For example, in humans such individuals would be suffering from dyslipidemia (e.g., a human with a HDL-C blood plasma level less than 60 milligrams/deciliter (mg/dL)). As a result, these individuals are at risk for atherosclerotic cardiovascular plaque formation that can result in cardiovascular diseases and circulatory disorders. Likewise, individuals without dyslipidemia but with an overall blood plasma level greater than 200 mg/dL (i.e., have hyperlipidemia) would also certainly benefit from treatment in accordance with the invention. The parameters for determining whether an individual is in need of treatment in accordance with the invention can be easily determined by a medical professional from routine clinical criteria.

In accordance with the present invention, the fluphenazine ester derivatives are administered to a mammal in any variety of drug delivery routes known in the art. Routes of administration include, but are not limited to, oral, parenteral (e.g., intravenous), intramuscular injection, intraperitoneal injection, subcutaneous injection, topical, transdermal, or the like. Preferred routes of administration include oral and subcutaneous injection.

The present invention also provides formulations for administering the above-described fluphenazine ester derivatives. The formulation included a therapeutically effective amount of the above-described fluphenazine ester derivatives and a pharmaceutically acceptable carrier. As will be apparent to one skilled in the art, the dosage form will be dependent on the route of administration. Depending on the intended mode of administration, the formulation can be in solid, semi-solid or liquid dosage forms, such as, for example, tablets, suppositories, pills, capsules, powders, liquids, suspensions, lotions, creams, gels, or the like. Preferably, the formulation is in unit dosage form suitable for single administration of a precise dosage. The formulations of the invention can include other medicinal agents, pharmaceutical excipients, adjuvants, diluents, etc. Actual methods of preparing such dosage forms are known, or will be apparent to those skilled in this art upon reviewing publicly available references such as Remington's Pharmaceutical Sciences (Martin, E. W., ed., 4th Ed., Mack Publishing Co., Easton, Pa.

Examples of pharmaceutical carriers are well known in the art. For example, in solid formulations, conventional nontoxic solid carriers include, but are not limited to, mannitol, lactose, starch, magnesium stearate, sodium saccharin, talc, cellulose, glucose, sucrose, magnesium carbonate, calcium carbonate, magnesium stearate, and the like. In liquid formulations, liquid carriers include, but are not limited to, water, saline, aqueous dextrose, glycerol, ethanol, sesame oil, DMSO, and the like, which can be used to form a solution or suspension. If desired, the formulations may also contain minor amounts of nontoxic auxiliary substances such as wetting or emulsifying agents, pH buffering agents and the like. Injectable formulations can be prepared in conventional forms such as either as liquid solutions or liquid suspensions. Likewise, injectable formulations may be in solid form suitable for solution or suspension in liquid prior to injection. A more recently revised approach for parenteral administration involves use of a slow release or sustained release system, such that a constant level of dosage is maintained (see, e.g., U.S. Pat. No. 3,710,795, which is incorporated by reference herein).

### EXAMPLES

The following non-limiting examples illustrate the advantageous use of fluphenazine ester derivatives to modulate HDL-C levels in mammals.

### Example 1

Fluphenazine 4-chlorophenoxyisobutyric acid ester (FZX-CETP-001) was synthesized following the protocol set forth in GB 2047694, which is herein incorporated by reference. The identity of fluphenazine 4-chlorophenoxyisobutyric acid ester was confirmed with the following spectroscopic and physical data—¹H nmr: 400 MHz, CDCl3: 1.57 (s, 6H), 1.95 (m, 2H), 2.10-2.55 (broad, 8H), 2.42 (t, J=6.0 Hz, 2H), 2.58 (t, J=6.0 Hz, 2H), 3.97 (t, J=6.0 Hz, 2H), 4.27 (t, J=6.0 Hz, 2H), 6.80 (d, J=8.1 Hz, 2H), 6.90-6.98 (m, 2H), 7.04 (s, 1H), 7.08-7.17 (m, 4H), 7.18 (d, J=8.1 Hz, 2H); m.p. (free base): 52-56° C.

### Example 2

The inhibitory activity of FZX-CETP-001 was determined in vitro using a commercially available CETP Inhibitor Drug Screening Kit (catalog # K602-100). The kit is available from Biovision Research Products, located in Mountain View, Calif. USA. Information regarding the kit is available at the following url addresses: http://www.biovisionlabs.com/cetp-pltp.html; and http://www.biovision.com/pdf/K602-100.pdf

Following the directions provided with the kit, FZX-CETP-001 was assayed for inhibitory activity. The results are listed in Table 1 below.

TABLE 1
Cholesterol Transfer Activity
(Average, N = 2)

| Assay | Fluorescence Units |
|---|---|
| CETP Enzyme (100% Active) | 17783 |
| CETP Enzyme + 100 μM FZX-CETP-0001 | 8312 |
| No CETP Enzyme (Fluorescence due to diffusion) | 9356 |

Based on the fluorescent data set forth in Table 1, FZX-CETP-0001 exhibited 100 percent inhibition of CETP at 100 μM. While the fluorescence units for FZX-CETP-0001 were less than the non-CETP containing sample, the discrepancy was attributed to experimental error.

### Example 3

CETP inhibitory activity and modulation of HDL-C to LDL-C ratios by FZX-CETP-001 was assayed in vivo using a transgenic mouse that expresses human CETP (hCETP). CETP protein does not normally occur in mice and thus transgenic mouse models expressing of hCETP had been developed. (Gautier et al., Apolipoprotein CI Overexpression Is Not A Relevant Strategy To Block Cholesteryl Transfer Protein (CETP) Activity In CETP Transgenic Mice, (2005) Biochem. J. 385:189-195). The mouse model, Apo*E3Leiden.hCETP mouse (http://www.tno.nl/kwaliteit_van_leven/markten/pharma/CETP%20mouse_website-tekst.pdf), is a recognized mouse model for testing the efficacy of candidate compounds plasma HDL-C-cholesterol and triglyceride levels as well as atherosclerosis. The mouse model is available for contract studies from TNO located in Zeist, The Netherlands. However, other CETP transgenic mouse models are well known in the art (see Jiang et al., Dietary Cholesterol Increases Transcription Of The Human Cholesteryl Ester Transfer Protein Gene In Transgenic Mice-Dependence On Natural Flanking Sequences, (1992) J. Clin. Invest. 90:1290-1295, which is incorporated herein by reference).

A test group of 36 female transgenic Apo*E3Leiden/hCETP mice were fed a high fat western-type diet (containing by weight 15% coconut oil and 0.1% cholesterol) to increase plasma cholesterol levels to around 10 mM. After three weeks the mice were subdivided into 6 groups of 6 mice each, matched for plasma cholesterol, triglycerides and age. After one week, the mice were treated with escalating dosages of FZX-CETP-001 (oral gavage: 0.1 mg/kg, 0.3 mg/kg, and 1 mg/kg; subcutaneous injection: 0.3 mg/kg, 1.0 mg/kg, and 3.0 mg/kg).

Oral gavage at each separate concentration was administered with an aqueous solution containing by volume 5% ethanol, 4% PEG-400, 1% cremaphor over a period of 1 week per concentration. At the end of each 1 week treatment period, blood samples were collected after a 4 hours fasting period. Mice treated by oral gavage were left alone for 1 week after each collection of blood samples.

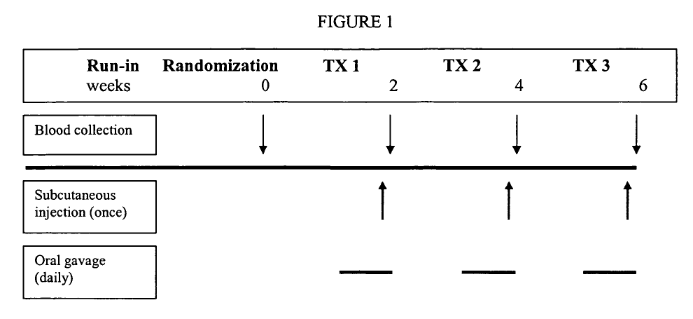

Subcutaneous depot injection was administered by injecting varying amounts of a 2.5-3.0% by weight to volume solution of powdered FZX-CETP-001 in sesame oil (powdered FZX-CETP-001, 25-30 mg; benzyl alcohol, 12 mg; sesame oil, 1 mL). For each subcutaneous depot injection experiment, the appropriate dose in sesame oil solution was injected once and blood samples were collected 24 hours after injection (at the end of a 4 hour fasting period). Mice treated by subcutaneous injection were left alone for 13 days after each collection of blood samples. The timeline for these two dosing regimens is shown in FIG. 1.

Blood samples from untreated test animals and from animals treated by gavage or subcutaneous depot injection were pooled separately and a lipoprotein profile was measured by AKTA fast protein liquid chromatography (FPLC)(http://www.gmi-inc.com/BioTechLab/Pharmacia%20AKTA.htm) to determine the level of cholesterol and phospholipid associated with HDL and LDL. (See Vrins C L J., Modulation of Gene Expression in the Liver: towards targeted correction of hyperlipidemia (Thesis), Leiden Univrsity Medical Center (2005); https://etc104.leidenuniv.nl/retrieve/1132/Proefschrift+Carlos+Vrins.pdf; see also Post et al., Cafestol Increases Serum Cholesterol Levels In Apolipoproteine*3-Leiden Transgenic Mice By Suppression Of Bile Acid Synthesis, (2000) Arterioscler. Thromb. Vasc. Biol. 20:1551-1556; Van Vlijmen et al., Modulation Of Very Low Density Lipoprotein Production And Clearance Contributes To Age And Gender-Dependent Hyperlipoproteinaemia In Apolipoprotein E3-Leiden Transgenic Mice, (1996) J. Clin. Invest. 97:1184-1192; which are incorporated herein by reference). Additionally, food intake levels (per group), body weights, cholesterol levels, and triglyceride levels, were monitored. Throughout most of the study, there was no statistically significant difference in the control parameters (food intake levels, body weights, cholesterol levels, triglyceride levels) except for the subcutaneous injections at doses at or above 1 mg/kg, for which the triglyceride level appeared to be slightly reduced.

The results from treating the mice with FZX-CETP-0001 by oral gavage and by subcutaneous depot injection are listed below in Tables 2 and 3, respectively. The numbers represent the percentage of lipoprotein type (HDL or LDL) and their associated cholesterol and phospholipid levels found in the AKTA-lipoprotein profile of the pooled blood samples relative to that found in the control (i.e., placebo treated) group.

TABLE 2

| Dosage | HDL | | LDL | |
|---|---|---|---|---|
| (mg/kg/day) | cholesterol | Phospholipids | cholesterol | Phospholipids |
| 0 | 100 | 100 | 100 | 100 |
| 0.1 | 204 | 128 | 81 | 81 |
| 0.3 | 121 | 136 | 94 | 92 |
| 1.0 | 269 | 129 | 80 | 96 |

TABLE 3

| Dosage | HDL | | LDL | |
|---|---|---|---|---|
| (mg/kg/day) | cholesterol | phospholipids | cholesterol | phospholipids |
| 0 | 100 | 100 | 100 | 100 |
| 0.3 | 286 | 141 | 100 | 71 |
| 1.0 | 277 | 173 | 78 | 56 |
| 3.0 | 1119 | 169 | 55 | 70 |

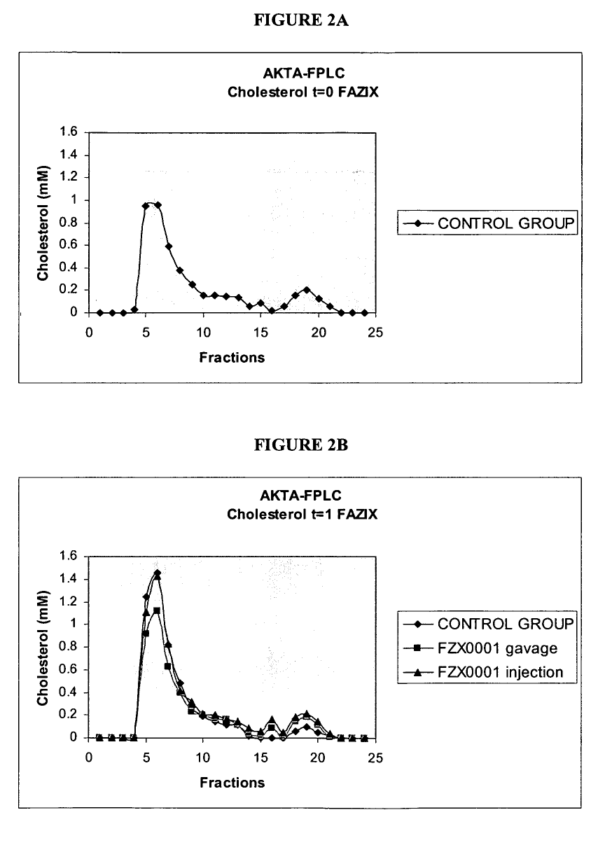

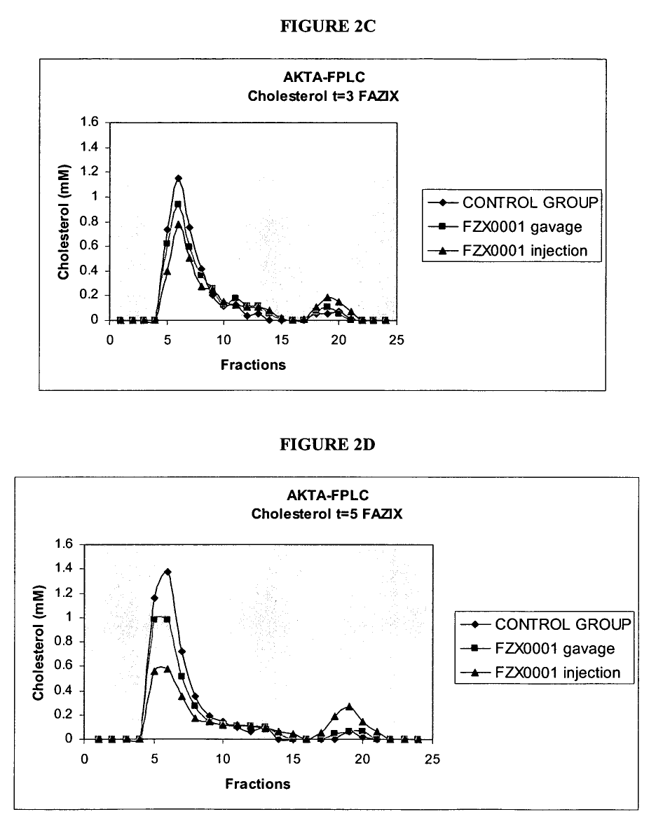

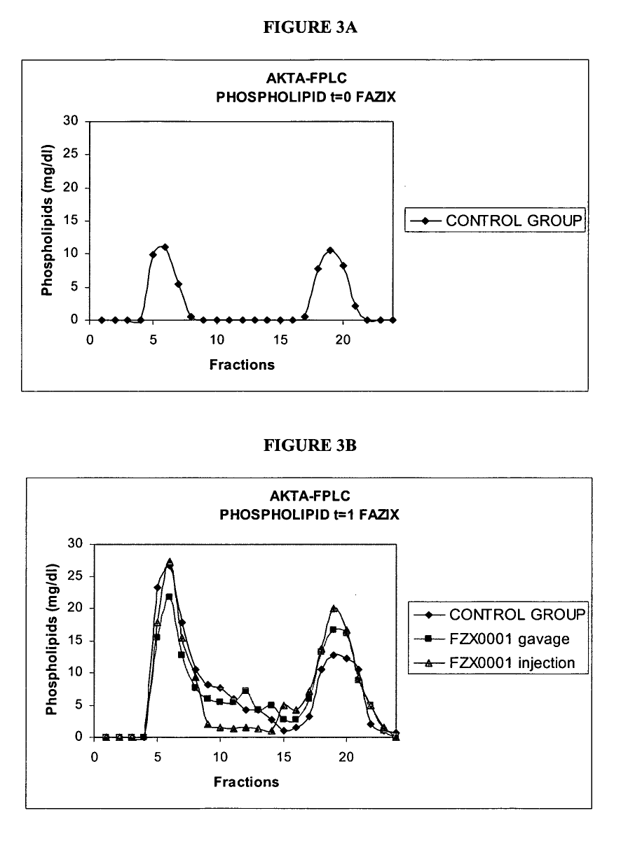

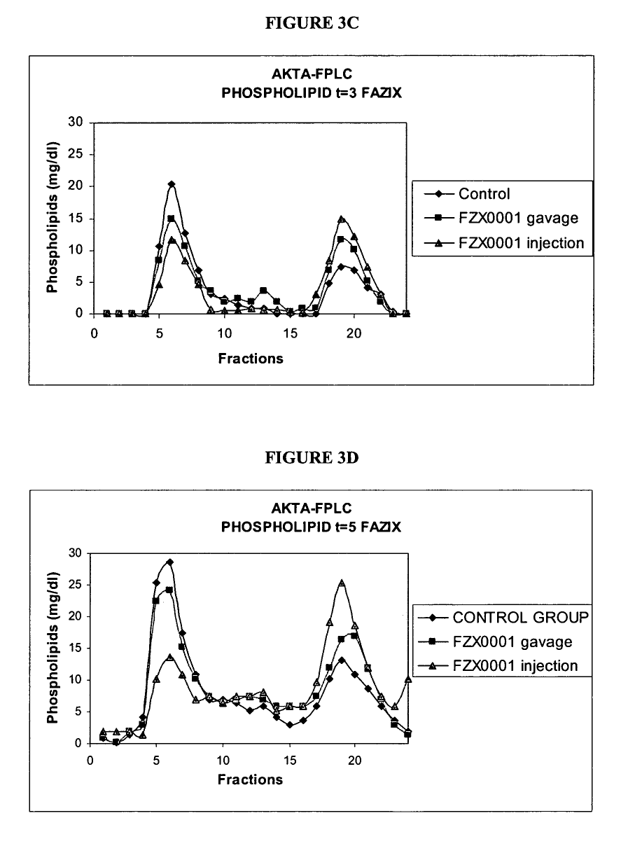

In addition, serum cholesterol and phospholipid profiles in samples collected from the three groups of mice (placebo -♦-, gavage -▪-, and subcutaneous injection -▴-) at each of the four time periods (t=0, t=1, t=3 and t=5) are graphically depicted in FIGS. 2A-2D and 3A-3D, respectively. Specifically, FIGS. 2A-2D are serum cholesterol profiles of blood sampled from the transgenic mice at progressively increasing doses of FZX-CETP-001. The first peak in these AKTA-FPLC chromatographic profiles (fractions 4-9) corresponds to LDL-C-cholesterol. The second peak (fractions 17-21) corresponds to HDL-C-cholesterol. FIG. 2A correspond to t=0 (i.e., placebo). FIGS. 2B-2D correspond to t=1, t=3 and t=5, which are sampling after 1 week of oral gavage treatment (0.1, 0.3, 1.0 mg/kg, respectively) or 1 day after a single subcutaneous depot injection (0.3, 1.0, 3.0 mg/kg/day respectively). As will be observed from FIGS. 2A-2D, the ratio of LDL-C level (total area under the curve) in treated to untreated mice decreases with increasing dose, whereas the ratio of HDL-C level in the treated to untreated mice increases.

Likewise, FIGS. 3A-3D are serum phospholipid profiles of blood sampled from the transgenic mice at progressively increasing doses of FZX-CETP-001. The first peak in these AKTA-FPLC chromatographic profiles (fractions 4-9) corresponds to LDL-C-phospholipid. The second peak (fractions 17-21) corresponds to HDL-C-phospholipid. FIG. 2A correspond to t=0 (i.e., placebo). FIGS. 2B-2D correspond to t=1, t=3 and t=5, which are sampling after 1 week of oral gavage treatment (0.1, 0.3, 1.0 mg/kg, respectively) or 1 day after a single subcutaneous depot injection (0.3, 1.0, 3.0 mg/kg/day respectively). As will be observed from FIGS. 2A-2D, the ratio of LDL-C phospholipid level (total area under the curve) in treated to untreated mice decreases with increasing dose, whereas the ratio of HDL-C level in the treated to untreated mice increases.

A comparison of corresponding measurements between the treated and untreated control group clearly indicates a marked increase in the HDL-C to LDL-C ratio even at the lowest tested dosage of 0.1 mg/kg/day by gavage. The data above demonstrates that fluphenazine ester derivatives, such as fluphenazine 4-chlorophenoxyisobutyric acid ester, are clearly effective in increasing HDL levels in mammals.

We claim:

1. A method of modulating the level of high-density-lipoprotein cholesterol in a mammal, which comprises administering to said mammal a therapeutically effective amount of an ester derivative of fluphenazine having the formula (I) 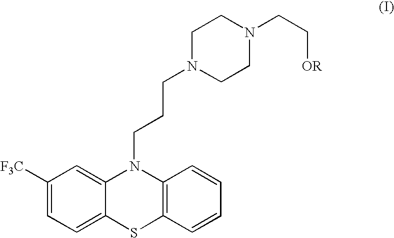
or a pharmaceutical acceptable salt thereof, wherein “R” is a 2 to 18 carbon atom-containing substituent with an acyclic carbonyl-terminated linker covalently bound to the fluphenazine moiety via the carbonyl terminus.

2. The method of claim 1, wherein R is a substituent including five to fourteen carbons atoms projecting a substantially planar face.

3. The method of claim 2, wherein R is a substituent having a substantially planar geometry except for the acyclic carbonyl linker.

4. The method of 3, wherein R is a cyclic ring structure being independently selected from the group consisting of a substituted or unsubstituted aromatic ring structure, a substituted or unsubstituted non-aromatic cyclic ring structure, a substituted or unsubstituted heterocyclic ring structure, and combinations thereof.

5. The method of claim 4, wherein said cyclic ring structure is independently selected from the group consisting of a monocyclic structure, a fused bicyclic structure and a fused tricyclic ring structure and combinations thereof.

6. The method of claim 1, wherein R is selected from the group consisting of 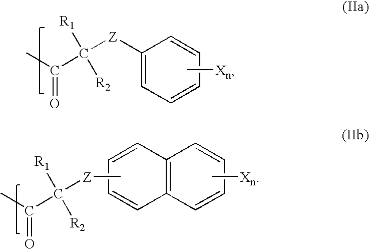
wherein R₁ and R₂ are independently hydrogen, methyl or ethyl, “Z” is methylene, oxygen, nitrogen or sulfur; “X” is independently a C₁₋₄ alkyl group, a halogen atom, a nitro group or C₁₋₄ alkyl ether groups, and “n” is 0 to 5 for formula (IIa) and 0 to 7 for formula (IIb), with the proviso that if “n” is greater than 1, “X” attached to the aromatic ring is same or different.

7. The method of claim 1, wherein R is selected from the group consisting of 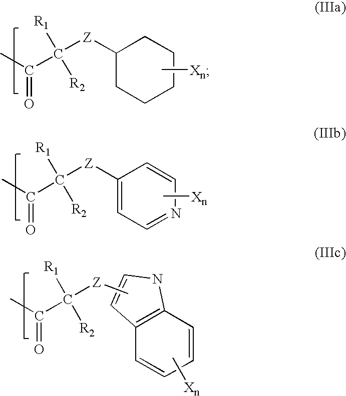
wherein R₁ and R₂ are independently hydrogen, methyl or ethyl, “Z” is methylene, oxygen, nitrogen or sulfur; “X” is independently a C₁₋₄ alkyl group, a halogen atom, a nitro group or C₁₋₄ alkyl ether groups, and “n” is 0 to 5 for formula (IIIa), 0 to 4 for formula (IIIb), and 0 to 7 for formula (IIIc), with the proviso that if “n” is greater than 1, “X” attached to the ring is the same or different.

8. The method of claim 1, wherein R is formula (IIa), R₁ and R₂ are methyl, Z is oxygen, X is chlorine and n is 1.

9. The method of claim 1, wherein said ester derivative of fluphenazine is fluphenazine 4-chlorophenoxyisobutyric acid ester.

10. The method of claim 1, wherein said mammal is in need of treatment.

11. The method of claim 1, wherein said mammal is a human.

12. The method of claim 1, wherein said ester derivative is administered in the amount of at least 0.1 mg/kg.

13. The method of claim 12, wherein said ester derivative is administered in an amount from 0.3 mg/kg.

14. The method of claim 1, wherein said ester derivative is administered in an amount that does not provide said mammal with a neuroleptic effect.

15. The method of claim 1, where said mammal exhibits at least a 10 percent increase in HDL-C levels.

16. The method of claim 1, where said mammal exhibits at least a 20 percent increase in HDL-C levels.

17. A pharmaceutical formulation for modulating the level of high-density-lipoprotein cholesterol in a mammal, which comprises:
a therapeutically effective amount of an ester derivative of fluphenazine having the formula (I) 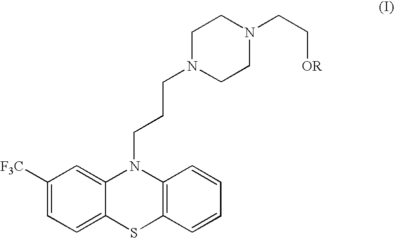
or a pharmaceutical acceptable salt thereof, wherein “R” is a 2 to 18 carbon atom-containing substituent with an acyclic carbonyl-terminated linker covalently bound to the fluphenazine moiety via the carbonyl terminus; and
a pharmaceutically acceptable carrier.

18. The formulation according to claim 17, wherein said ester derivative of fluphenazine is fluphenazine 4-chlorophenoxyisobutyric acid ester.
